(19) United States
(12) Patent Application Publication
(10) Pub. No.: US 2023/0148902 A1
(43) Pub. Date: May 18, 2023

(54) VOC CAPTURE AND STORAGE SYSTEM AND METHOD OF USE

(71) Applicant: Jeffrey R. Mitchell, University City, MO (US)

(72) Inventor: Jeffrey R. Mitchell, University City, MO (US)

(21) Appl. No.: 17/974,489

(22) Filed: Oct. 26, 2022

Related U.S. Application Data
(60) Provisional application No. 63/263,054, filed on Oct. 26, 2021.

(51) Int. Cl. A61B 5/097

(52) U.S. Cl. CPC A61B 5/097

(57) ABSTRACT

A method of capturing volatile organic compounds entrained in respiratory gases of a subject. The method includes fluidly communicating the respiratory gases of the subject with a passage of a system. The passage extends from the entrance at an upstream end of the passage to an exit at a downstream end of the passage. The system includes a particle collector positioned in the passage. The subject is allowed to exhale while the passage of the system is in fluid communication with the respiratory gases of the subject. After a predetermined event, the passage of the system is fluidly isolated from the respiratory gases of the subject.

### CROSS-REFERENCE TO RELATED APPLICATION

The present application claims benefit to the extent permitted by law of U.S. Patent Application 63/263,054, entitled, “VOC Capture System and Method of Use,” filed on Oct. 26, 2021, which is hereby incorporated by reference in its entirety.

### BACKGROUND

The invention relates generally to devices and methods for capturing and storing small particles and more specifically to system and method for capturing and storing particles including volatile organic compounds (VOCs) from a subject's breath.

Common devices for capturing and storing small particles include test tubes, swabs, and adhesive films. Frequently, the particles desired to be collected are VOCs found in a subject's nose or throat. In one example of collecting these small particles, a swab is rubbed against a selected area of the subject to dislodge the small particles and retain those particles on or between fibers forming the swab. Once the particles are collected, the swab is sealed in a vial or other container for storage. Later, the swab and collected particles are removed from the container for use.

One of the problems with using a swab or other collection device results from the locations where the particles are found. For example when the particles of interest are in a subject's nose, the particles are usually concentrated a few inches inside nose and toward the back of the nasal cavity. When a swab is inserted to this location, most subjects experience discomfort. Another problem inherent with collecting particles with a swab is that the swab is susceptible to contacting mucus and debris in the nose when being inserted and withdrawn. During insertion, contact with these untargeted materials blocks the swab fibers from picking up desired particles once the swab reaches the target site, and during removal, contact with these materials often dislodges collected particles from the swab fibers. Thus, swabs are not optimally suited for collecting particles. Moreover, after the target particles are collected on a swab, the particles frequently must be separated from the swab fibers and untargeted materials before they can be studied. The separation processes further decrease the effectiveness and efficiency of collecting particles with a swab.

Many other collection systems suffer from similar limitations. Although improvements have been made in particle capture and storage systems further improvements are needed.

### SUMMARY

In one aspect, the present disclosure includes a method of capturing volatile organic compounds entrained in respiratory gases of a subject. The method comprises the steps of fluidly communicating the respiratory gases of the subject with a passage of a system. The passage extends from the entrance at an upstream end of the passage to an exit at a downstream end of the passage. The system includes a particle collector positioned in the passage. The subject is allowed to exhale while the passage of the system is in fluid communication with the respiratory gases of the subject. After a predetermined event, the passage of the system is fluidly isolated from the respiratory gases of the subject.

In another aspect, the present disclosure includes a system for capturing and storing particles entrained in fluid. The system comprises a tubular body having an inner surface defining a passage extending from an entrance at an upstream end of the passage to an exit at a downstream end of the passage. A collector lines the inner surface of the body. The collector is adapted to collect at least part of the particles entrained in the fluid flowing through the passage from the entrance to the exit and to retain at least a portion of the collected particles for later use.

In yet another aspect, the present disclosure includes a system for capturing and storing volatile organic compounds entrained in respiratory gases of a person. The system comprises a tubular body having an inner surface defining a passage extending from an entrance at an upstream end of the passage to an exit at a downstream end of the passage. A multiplicity of filaments are positioned in the passage of the body adapted to capture volatile organic compounds entrained in respiratory gases exhaled by the person. Each of the filaments extends from the inner surface of the tubular body.

Other aspects of the present disclosure will be apparent in view of the following description and claims.

### BRIEF DESCRIPTION OF THE DRAWINGS

FIG. 1 is a schematic cross-sectional side elevation of a first example of a VOC capture system;

FIG. 2 is a schematic cross-sectional side elevation of a second or alternative example of a VOC capture system; and

FIG. 3 is a flowchart illustrating a method of collecting VOCs.

Corresponding reference characters indicate corresponding parts throughout the drawings.

### DETAILED DESCRIPTION OF THE DRAWINGS

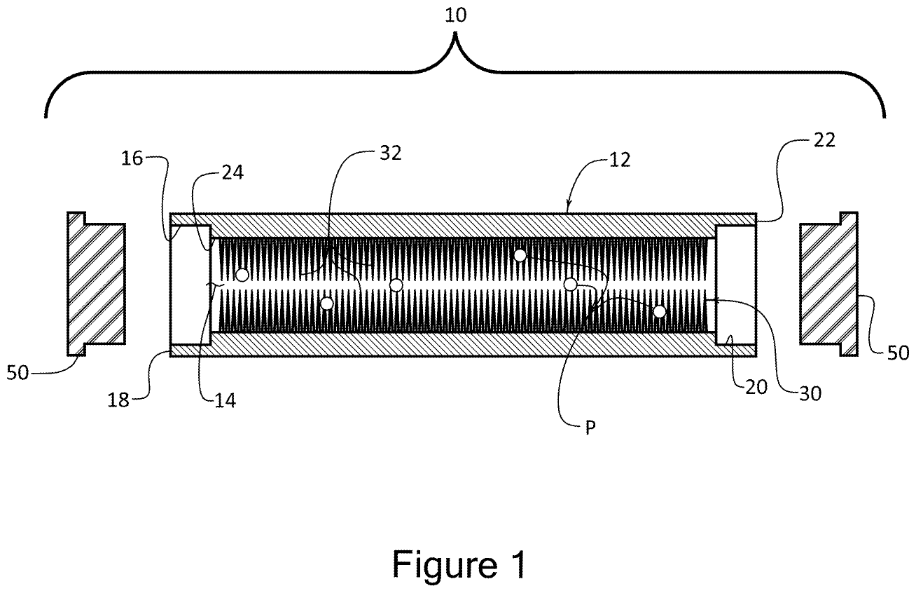

Referring to the drawings, and more particularly to FIG. 1, a system for collecting and retaining particles (e.g., capturing volatile organic compounds (VOCs)) is designated in its entirety by the reference number 10. As will be explained, the system 10 is also adapted for storing the captured particles. Although the primary focus of the system is collecting VOCs from a subject's respiratory gases, it is envisioned that similar systems could be used to collect particles from fluids generally. The illustrated system 10 comprises a tubular body, generally designated by 12 having a passage 14 extending from an entrance 16 at an upstream end 18 of the passage to an exit 20 at a downstream end 22 of the passage. The passage 14 is defined by an inner surface 24 of the body 12 having a collector, generally designated by 30. In the illustrated example, the collector 30 comprises a plurality of filaments 32 extending inward into the passage 14 from the inner surface 24 of the body 12. As will be appreciated, the passage 14 permits fluid flow through the body 12 from the upstream end 18 to the downstream end 22. The filaments 32 are adapted to collect particles P or VOCs carried by the fluid passing through the passage 14. Although the filaments may be made from other suitable materials such as cotton, felted synthetic material, woven or knitted fabric, or other suitable textiles, the illustrated filaments 32 are made from fibers comprising a suitable synthetic polymer. It is envisioned that the filaments 32 may be treated with an adhesive coating or electrostatic charge to enhance their collection and retention properties. Even though the illustrated passage 14 is shown as extending in a straight line between the upstream end 18 and the downstream end 22, the passage may have other, non-linear, shapes. Further, it is envisioned the passage 14 may have a cross section that varies in size and shape between the upstream end 18 and the downstream end 22 of the passage.

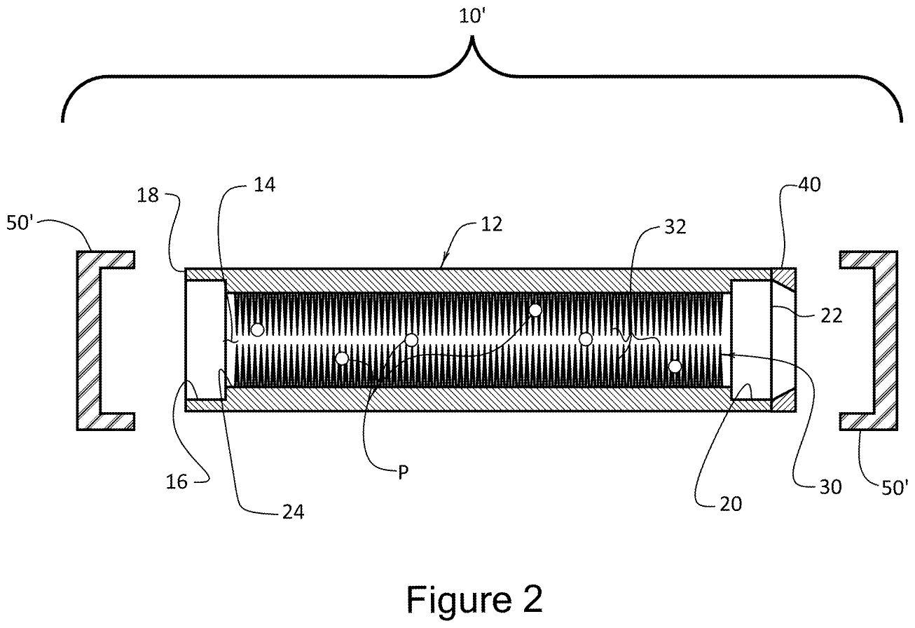

In a second example shown in FIG. 2, the system 10′ is generally identical to the system of the first example shown in FIG. 1 except the system of the second example has a flowmeter 40 positioned in fluid communication with the passage 14 so the flowmeter measures the fluid passing through the passage. Although the flowmeter 40 may be located at other locations, the illustrated flowmeter is mounted on the body 12 immediately upstream from the entrance 16. It is envisioned the flowmeter 40 may be made to separate from the system 10′ so the same flowmeter may be used with multiple systems 10. The flowmeter 40 measures fluid flow through the passage 14 allowing a volume of gas having passed through the flowmeter to be calculated. Thus, the flowmeter 40 may enable a user to know when a predetermined volume of fluid has passed through the passage 14 to ensure target particles are captured by the by the collector if they are present in sufficient numbers. Measuring passing volume also enables a user to estimate a density of entrained particles P.

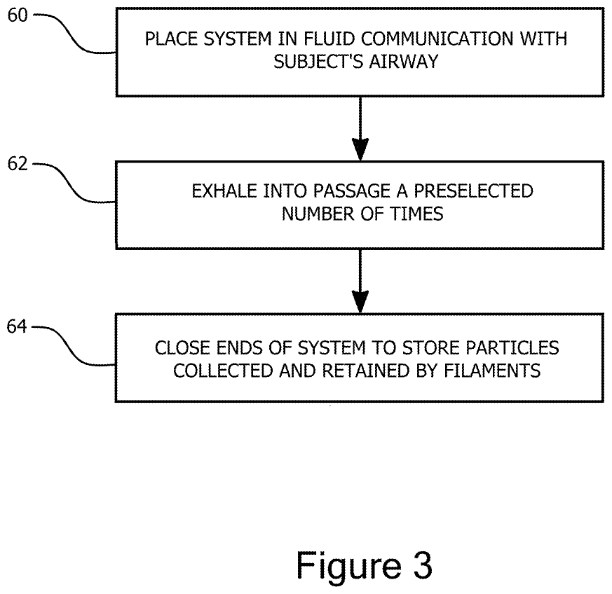

To use the system 10, the entrance 16 is placed in fluid communication with fluid (e.g., gas) emitted from a subject as shown in FIG. 3 at step 60. Preferably, the selected fluid passes over a site having target particles or VOCs and picks up or flushes the particles from the target so particles become entrained in the fluid stream. The fluid stream and entrained particles are directed into the system 10 through the entrance 16. In the case of target particles potentially being present in a subject's lungs, the particles are likely to be present in exhaled respiratory gases. Thus, a subject may hold the upstream end 18 of the system 10 between their lips and exhale a preselected number of times into the entrance 16 of the system. As the subject exhales the respiratory gases travel through the passage 14 with entrained particles P (FIG. 1). As illustrated at step 62 of FIG. 3, the filaments 32 collect and retain at least some of the particles P traveling through the passage 14 before the gas leaves the system through the exit 20 at the downstream end 22 of the system. Alternatively, medical personnel may position the system 10 in fluid communication with respiratory equipment (not shown) during a selected number of exhalations or for a selected period of time to collect particles P on or between the filaments 32. After capturing the particles, a user may store the particles P in the collector 30 by plugging the entrance 16 and the exit 20 with corresponding caps or plugs 50 as shown in step 64 of FIG. 3. The plugs 50 are shown in FIGS. 1 and 2 as forming part of the system 10 or 10′. As will appreciated the system 10 or 10′ may be shipped with the plugs 50 positioned on the upstream end 18 and downstream end 22. A user may remove the plugs 50 prior to use and reinstall them once collection is complete to store sample until used. With the plugs in position, the passage 14 is isolated from contaminates prior to use and after use.

Collecting respiratory samples using either of the described systems 10, 10′ is significantly less intrusive and much simpler than collecting samples many conventional collection devices such as swabs. Further, the collection procedure is much more repeatable than prior art collection devices. Therefore several benefits and advantages are provided by the systems 10, 10′.

The particular examples, as well as the concepts and principles described in the foregoing text and shown in the accompanying drawings are not intended to limit the scope of the claims that follow. It is intended that all matter contained in the above description or shown in the accompanying drawings shall be interpreted as illustrative and not in a limiting sense. Further, it is envisioned that various changes could be made to the constructions and processes described above without departing from the scope of the claims. As far as the description and accompanying drawings disclose additional subject matter that is not within the scope of the claims, these additional disclosures are not dedicated to the public and the right to file additional claims and additional applications is reserved.

1. A method of capturing volatile organic compounds entrained in respiratory gases of a subject, said method comprising the steps of:
fluidly communicating the respiratory gases of the subject with a passage of a system, said passage extending from the entrance at an upstream end of the passage to an exit at a downstream end of the passage, said system including a particle collector positioned in the passage;
allowing the subject to exhale while the passage of the system is in fluid communication with the respiratory gases of the subject; and
after a predetermined event, fluidly isolating the passage of the system from the respiratory gases of the subject.

2. A method as set forth in claim 1, wherein the predetermined event comprises a preselected number of subject exhalations.

3. A method as set forth in claim 1, wherein the predetermined event comprises a preselected volume of gas exhaled by the subject.

4. A method as set forth in claim 1, further comprising step of closing the entrance and exit of the passage after the passage of the system is fluidly isolated from the respiratory gases of the subject.

5. A method as set forth in claim 4, further comprising step of opening the entrance and exit of the passage before fluidly communicating the respiratory gases of the subject with the passage of the system with the respiratory gases of the subject.

6. A system for capturing and storing particles entrained in fluid, the system comprising:
a tubular body having an inner surface defining a passage extending from an entrance at an upstream end of the passage to an exit at a downstream end of the passage; and
a collector lining the inner surface of the body, the collector being adapted to collect at least part of the particles entrained in the fluid flowing through the passage from the entrance to the exit and retain at least a portion of the collected particles for later use.

7. A system as set forth in claim 6, wherein the collector comprises a multiplicity of filaments positioned in the passage.

8. A system as set forth in claim 7, wherein each of the filaments of said multiplicity of filaments extends from the inner surface of the tubular body.

9. A system as set forth in claim 8, wherein said multiplicity of filaments is adapted to capture volatile organic compounds entrained in respiratory gases of a subject.

10. A system as set forth in claim 7, further comprising a pair of plugs, one plug of said pair of plugs being sized and shaped to close the entrance of the passage and another plug of said pair of plugs being sized and shaped to close an exit of the passage.

11. A system as set forth in claim 7, further comprising a flowmeter in fluid communication with the passage for measuring fluid flow through the passage.

12. A system for capturing and storing volatile organic compounds entrained in respiratory gases of a person, said system comprising:
a tubular body having an inner surface defining a passage extending from an entrance at an upstream end of the passage to an exit at a downstream end of the passage; and
a multiplicity of filaments positioned in the passage of the body adapted to capture volatile organic compounds entrained in respiratory gases exhaled by the person, each of the filaments of said multiplicity of filaments extending from the inner surface of the tubular body.

13. A system as set forth in claim 12, wherein the upstream end of the passage is sized and shaped for being held between lips of a mouth of the person.

14. A system as set forth in claim 12, further comprising a pair of plugs, one plug of said pair of plugs being sized and shaped to close the entrance of the passage and another plug of said pair of plugs being sized and shaped to close an exit of the passage.

15. A method of using a system as set forth in claim 12 comprising the steps of:
holding the upstream end of the passage between lips of the mouth;
exhaling into the entrance of the passage a preselected number of times; and
after the preselected number of exhalations, removing upstream end of the system from between the lips of the mouth.

16. A method as set forth in claim 15, further comprising the step of capping the entrance and the exit of the passage after removing upstream end of the system from between the lips.

17. A method as set forth in claim 15, further comprising step of uncapping the entrance and the exit of the passage before holding the upstream end of the passage between lips of the mouth.
